(19) United States
(12) Patent Application Publication
(10) Pub. No.: US 2025/0224513 A1
(43) Pub. Date: Jul. 10, 2025

(54) METHOD AND APPARATUS FOR ULTRASONIC EVALUATION OF AN ISOLATED ORGAN

(71) Applicants: INSTITUT NATIONAL DE LA SANTÉ ET DE LA RECHERCHE MÉDICALE, Paris (FR); INSTITUT NATIONAL DE RECHERCHE POUR L'AGRICULTURE, L'ALIMENTATION ET L'ENVIRONNEMENT, Paris (FR); ECOLE SUPÉRIEURE DE PHYSIQUE ET DE CHIMIE INDUSTRIELLES DE LA VILLE DE PARIS, Paris (FR); CENTRE NATIONAL DE LA RECHERCHE SCIENTIFIQUE, Paris (FR); UNIVERSITE CLAUDE BERNARD LYON 1, Villeurbanne (FR)

(72) Inventors: Mathieu PERNOT, Paris (FR); Mickael TANTER, Paris (FR); Oscar DEMEULENAERE, Paris (FR); Clément PAPADACCI, Paris (FR); Olivier PEREIRA PEDREIRA, Paris (FR); René FERRERA, Bron (FR)

(21) Appl. No.: 18/853,607

(22) PCT Filed: Apr. 11, 2023
(86) PCT No.: PCT/EP2023/059456

§ 371 Date: Oct. 2, 2024

(30) Foreign Application Priority Data
Apr. 11, 2022 (EP) 22305523.7

(51) Int. Cl. G01S 15/89

(52) U.S. Cl. CPC G01S 15/8934; G01S 15/8993

(57) ABSTRACT

Organ transplantation remains the only definitive therapeutic solution for many pathologies, but the number of currently available grafts is largely insufficient. A new approach is proposed to quantitatively evaluate isolated organs by ultrasound, which enables to safely admit more isolated organs as grafts available for transplantation. The isolated organ (2) is received in an organ preservation container (3) made of ultrasound transparent material, and the isolated organ is imaged by an ultrasound imaging probe (6) through the container (3). The ultrasound image of the isolated organ is used to determine a quantitative index representing viability of the isolated organ.

### TECHNICAL FIELD

The present disclosure relates to methods and apparatuses for ultrasonic evaluation of isolated organs.

### PRIOR ART

Organ transplantation remains the only definitive therapeutic solution for many cardiac, renal or hepatic pathologies. However, the number of grafts available for organ transplantation is largely insufficient. Many additional grafts could be available with broader criteria for procurement and preservation. Expanding these criteria requires new means to assess organ integrity and functionality before transplantation.

Once the organ is harvested, the current main approach to evaluation relies on the donor's clinical parameters and a qualitative assessment of the graft by the surgeon based on a few indicators such as visual appearance, color and manual palpation. This evaluation remains highly subjective and does not include any functional parameters. Quantitative evaluation of tissue or vascular properties would allow screening of all available grafts and broaden the number of grafts acceptable for organ transplantation. Two sources of cardiac grafts (currently not exploited) are concerned for an initial assessment of their viability: grafts with a long shelf life (>4-6 h) and grafts harvested after warm ischemia (so-called Maastricht-III grafts).

Moreover, current needs in the field of organ preservation tends to increase the storage time (grafts transported on long distances). The quantitative evaluation of grafts is all the more important to define a conservation threshold adapted to each graft. Finally, the current explosion in the field of regenerative medicine requires tools for anatomical and functional evaluation of organs, especially tissue and vascular properties. Modalities such as MRI or CT are not adapted to graft characterization due to cost and time constraints. Conversely, conventional ultrasound imaging allows a cheap and rapid evaluation, but does not provide quantitative data.

### OBJECTS

The present specification proposes a new approach to characterize isolated organs by ultrasound, enabling to quantitatively evaluate cardiac or other grafts and if need be, monitor tissue and vascular parameters during organ preservation.

One object of the present disclosure is thus a method for ultrasonic evaluation of an isolated organ from a human or animal received in an organ preservation container made of an ultrasound transparent material, said method comprising:
holding an ultrasound imaging probe against said organ preservation container to perform ultrasound imaging of said isolated organ through said organ preservation container;
imaging said isolated organ by an ultrasound imaging system communicating with said ultrasound imaging probe, to obtain at least one ultrasound image of said isolated organ,
automatically determining at least one quantitative index representing viability of said isolated organ, said at least one quantitative index being calculated from anatomical, tissue or vascular parameters obtained from said at least one ultrasound image.

In embodiments of the above method, one may further use one or several of the following features and any combination thereof:
said ultrasound imaging probe is automatically moved by a holder device during said imaging, to obtain different images of said isolated organ as said ultrasound imaging probe is moved;
predetermined anatomic areas of said isolated organ are automatically identified from said different images and said at least one quantitative index is automatically computed in at least one area of interest among said predetermined anatomic areas;
said at least one ultrasound image of said isolated organ is either a 2D image or a 3D image;
said at least quantitative index is determined for several predetermined areas of said isolated organ, and shown on a parametric map of said isolated organ;
said isolated organ is chosen among a heart, a kidney and a liver;
said at least one quantitative index is a rheological index determined by elastography through said ultrasound imaging probe and ultrasound imaging system;
said isolated organ includes fibers and said at least one quantitative index includes a rheological elasticity parameter measured along said fibers and a rheological elasticity parameter measured perpendicular to said fibers;
said rheological index is chosen among stiffness, propagation speed of shear waves, fractional anisotropy, shear modulus, Young's modulus, viscosity, elastic anisotropy;
said at least one quantitative index is representative of vascular flows in the isolated organ under perfusion;
said at least one quantitative index is chosen among Doppler signal (e.g. Power Doppler, pulsed Doppler), dimensions of vessels, blood velocity, blood flow and blood volume;
said at least one quantitative index is representative of vascular network geometry in said isolated organ under perfusion (for instance obtained by ultrasound localization microscopy of said isolated organ, in particular with circulation of microbubbles or other contrast agent in said vascular network);
said isolated organ is perfused with a solution including a contrast agent adapted to enhance contrast for ultrasound imaging;
said at least one quantitative index is chosen among dimensions of blood vessels, in particular micro-vessels, density and tortuosity of the vascular network;
said at least one quantitative index is a property related to ultrasound backscatter, such as backscattered tensor imaging or backscattered energy.

Several of the above quantitative indexes can be cumulatively used.

In one embodiment, said organ preservation container may be made of an ultrasound transparent and rigid material.

In one embodiment, said organ preservation container may has a geometric shape adapted to perform multidirectional imaging of the isolated organ.

Besides, another object of the present disclosure is an apparatus for ultrasonic evaluation of an isolated organ from a human or animal, comprising:
an organ preservation container made of an ultrasound transparent material and adapted to contain said isolated organ;
an ultrasound imaging probe;
a holder device for holding the ultrasound imaging probe against said organ preservation container to perform ultrasound imaging of said isolated organ through said organ preservation container;
an ultrasound imaging system communicating with said ultrasound imaging probe to obtain at least one ultrasound image of said isolated organ, said ultrasound imaging system being adapted to determine at least one quantitative index representing viability of said isolated organ, said at least one quantitative index being calculated from anatomical, tissue or vascular parameters obtained from said at least one ultrasound image.

In embodiments of the above apparatus, one may further use one or several of the following features and any combination thereof:
said holder device is adapted to automatically move said ultrasound imaging probe, said ultrasound imaging system communicating with said holder device and being adapted to control said ultrasound imaging probe to obtain different images of said isolated organ as said ultrasound imaging probe is moved;
said at least one quantitative index is a rheological index and said ultrasound imaging system is adapted to determine said rheological index by elastography;
said at least one quantitative index is representative of vascular flows in the isolated organ;
said at least one quantitative index is representative of vascular network geometry in said isolated organ;
said at least one quantitative index is a property related to ultrasound backscatter;
said organ preservation container is made of an ultrasound transparent and rigid material.
said organ preservation container has a geometric shape adapted to perform multidirectional imaging of the isolated organ.

### BRIEF DESCRIPTION OF THE DRAWINGS

Other features and advantages will appear from the following description of one embodiment, given by way of non-limiting example, with regard to the drawings.

In the drawings:

FIG. 1 is a perspective view of an apparatus for ultrasonic evaluation according to one embodiment;

FIG. 2 is a block diagram of the apparatus of FIG. 1;

FIG. 3 shows ultrasound reconstruction of the heart (reconstruction of the short-axis view from a scan performed on each side of the box; long-axis reconstruction on the right ventricle (RV) and on the left ventricle (LV). On each reconstruction, the cardiac structures were labeled allowing correct identification of the structures);

FIG. 4A shows a map of the shear rate (shear wave velocity) of the LV wall of a porcine heart just after collection and after 24 h of storage;

FIG. 5A shows a map of the shear rate (shear wave velocity SWV) of LV wall of ischemic hearts just after harvesting and after 24 hours of storage;

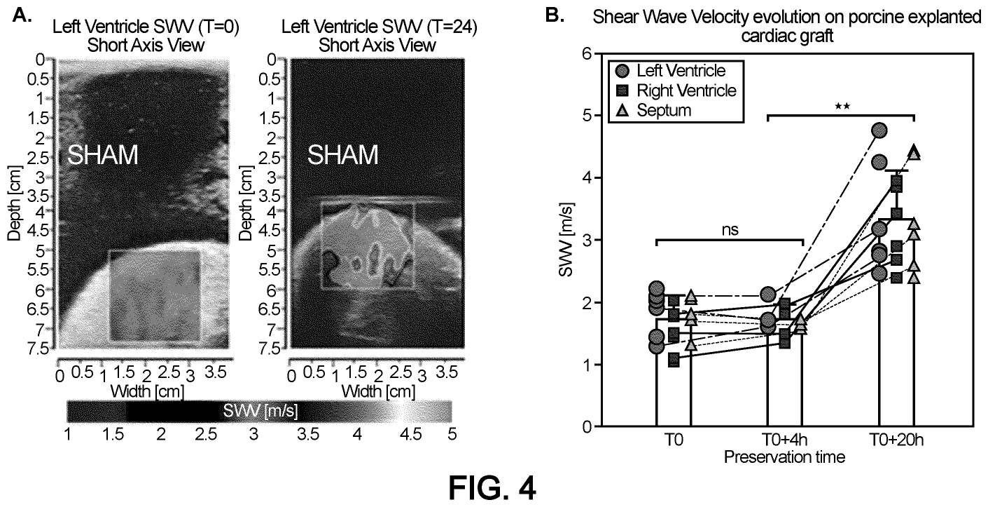

FIG. 4B shows the evolution of global stiffness (shear wave velocity SWV) of porcine control hearts during storage, as a function of time;

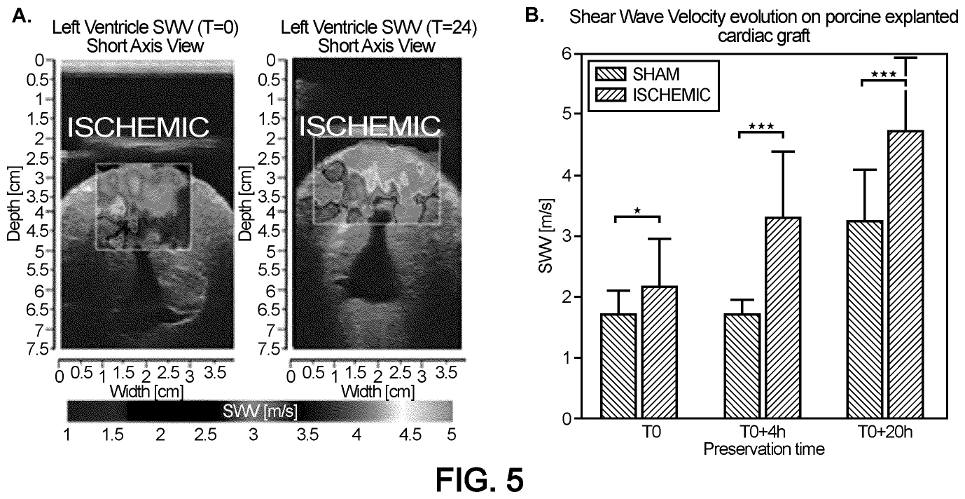

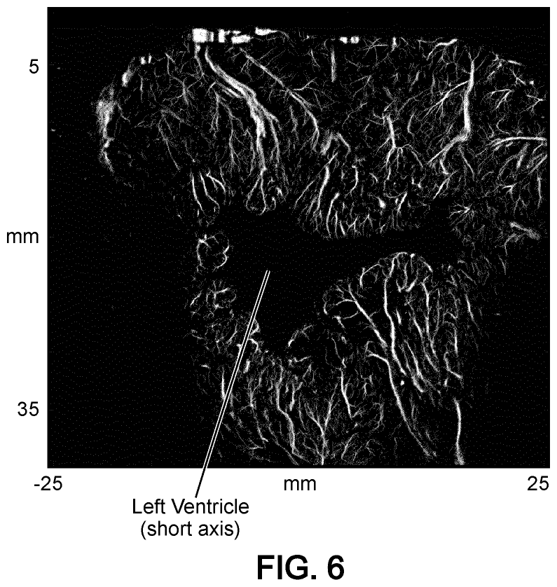

FIG. 5B shows the evolution of global stiffness of ischemic porcine hearts versus control porcine hearts (sham) during storage;

FIG. 6 shows an image of ultrasound localization microscopy on a perfused porcine heart (short axis section of the LV).

### MORE DETAILED DESCRIPTION

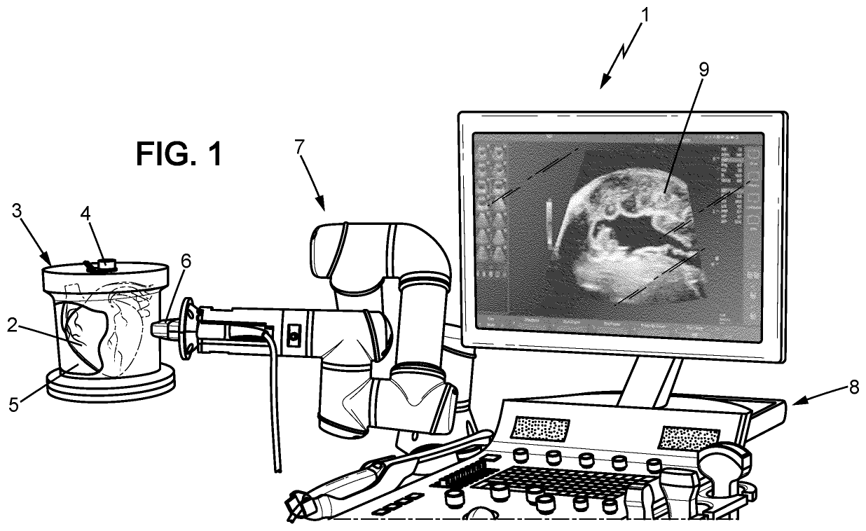

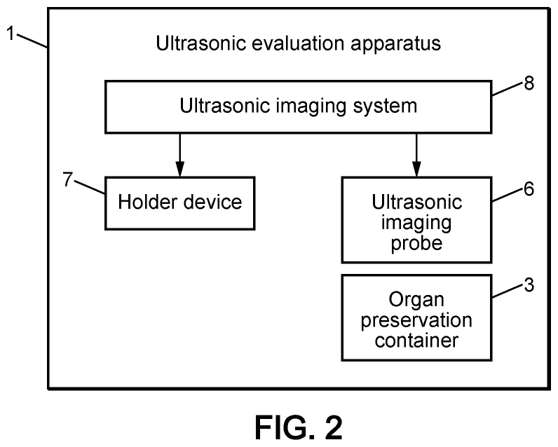

FIGS. 1 and 2 show an apparatus 1 for ultrasonic evaluation according to one embodiment.

The apparatus 1 is for evaluation of an isolated organ 2 from a human or animal, which is meant to be used as a graft.

Isolated organ 2 may be for instance a heart or a kidney or a liver, or another organ.

The apparatus 1 comprises an organ preservation container 3 made of an ultrasound transparent material and adapted to contain said isolated organ 2. The organ preservation container 3 is a rigid box in the illustrated example, but could be any other packaging having rigid, semi-rigid or flexible walls made of a material which is transparent to ultrasounds.

The ultrasound transparent material may be for instance polymethylpentene, e.g. the polymethylpentene sold under trademark TPX®.

In a preferred embodiment, the box comprises peripheral walls made of an ultrasound transparent and rigid material. The peripheral wall has a geometric shape adapted to perform an ultrasound imaging of the whole isolated organ 2 under different directions. In particular, the shape is adapted to perform a multidirectional ultrasound imaging.

As illustrated in FIG. 1, the peripheral wall of the preservation container may have a cylindrical shape, in particular a cylinder having a circular basis, allowing an ultrasound probe to move peripherally along the wall to image the organ under different directions, in particular different directions oriented radially with respect to the central axis of the cylinder. The probe may also be moved axially along the axis of the cylinder, to take images of the organ at different heights along the axis.

The use of rigid walls permits to avoid mechanical deformation of the organ during ultrasound imaging. This avoids risks of mechanical damage to the organ and improves accuracy of the imaging and measures done through the ultrasound probe, as the organ is in the same state during successive images of the organ (for instance, in the case of a cylindrical container, the images taken in different radial directions and at different heights along the axis of the cylinder).

Isolated organ 2 may be immersed in any graft preservation solution in organ preservation container 3.

Organ preservation container 3 may have fluid connectors 4, 5 for perfusion of isolated organ 2, as known in the art.

Organ preservation container 3 is sealed for conservation of isolated organ 2 and is openable for grafting isolated organ 2.

The apparatus 1 further includes an ultrasound imaging probe 6, which may be of any type known in the art.

Ultrasound imaging probe 6 may for instance include a linear array of ultrasound transducers for 2D ultrasound imaging, or a 2D array of ultrasound transducers for 3D ultrasound imaging. In a variant or in addition, ultrasound imaging probe 6 may include transducers as described for instance in WO2015/114232A1.

The apparatus 1 further includes a holder device 7 for holding ultrasound imaging probe 6 against said organ preservation container 3 to perform ultrasound imaging of isolated organ 2 through said organ preservation container 3.

Some gel may be used to facilitate transmission of ultrasounds between ultrasound imaging probe 6 and organ preservation container 3, as known in the art.

Holder device 7 may be a robotic arm. Said robotic arm may be for instance a 6-axis robotic arm but may be of any other type.

Holder device 7 may be usable to automatically move ultrasound imaging probe 6 during imaging, to obtain successive images of different portions of isolated organ 2 or of the whole isolated organ 2 under different directions as said ultrasound imaging probe 6 is moved.

The apparatus 1 further includes an ultrasound imaging system 8, which can be or include a computer system having at least one display screen 9 and input interfaces (not shown) such as inter alia a keyboard and a mouse for a user.

Ultrasound imaging system 8 communicates with ultrasound imaging probe 6 to obtain at least one ultrasound image of isolated organ 2, in any way which is known in the art.

Ultrasound imaging system 8 may also communicate with holder device 7 and control said holder device to automatically move ultrasound imaging probe 6 by holder device 7 and obtain successive ultrasound images, as explained above.

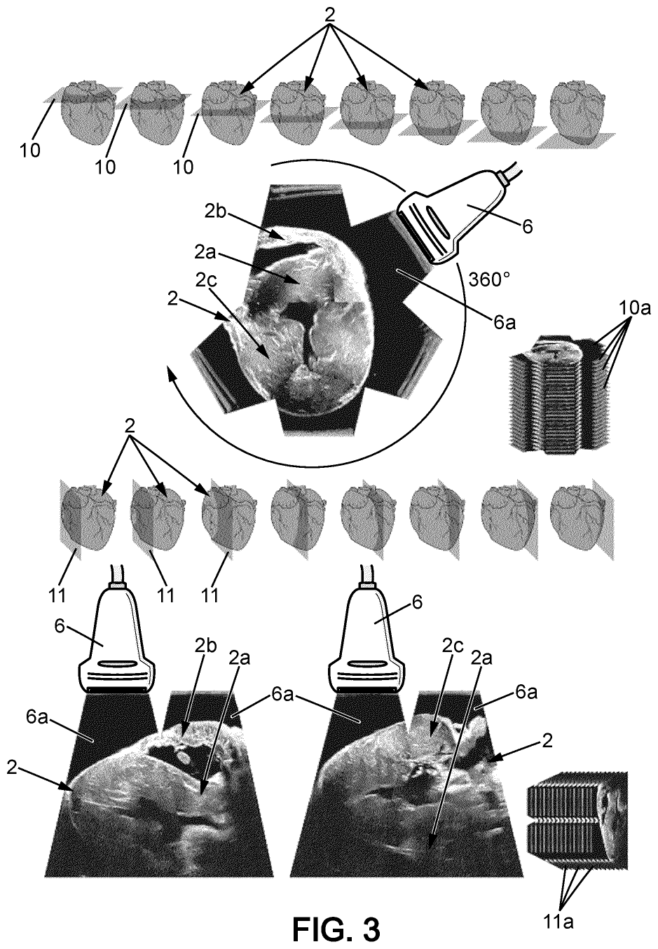

For instance, as illustrated in FIG. 3 for the case where the isolated organ 2 is a heart, ultrasound imaging probe 6 may include a linear array of transducers and ultrasound imaging system 8 may control holder device 7 and ultrasound imaging probe 6 so as to take:
a series of short axis ultrasound images 10a taken in successive parallel planes 10; in each plane 10, ultrasound imaging probe may be turned around organ preservation container 3 so that the ultrasound beam 6a from ultrasound imaging probe 6 takes several 2D images at several predetermined angles around organ preservation container 3;
a series of long axis ultrasound images 11a taken in successive parallel planes 11; in each plane 11, ultrasound imaging probe 6 may be moved relative organ preservation container 3 so that the ultrasound beam 6a from ultrasound imaging probe 6 takes a 2D image on the side of the RV and a 2D image on the side of the LV.

The 2D images are then used to reconstruct an anatomical 3D image of the complete heart.

The automatic scan of the heart as explained above enables to identify, automatically or manually, heart structures such as the septum 2a, the RV 2b and the LV 2c.

In a preferred embodiment, one or several areas of interest are predetermined automatically from the 2D images. Thus, the proposed method advantageously allows an accurate and rapid determination of the areas of interest. The proposed method is thus more reproducible, and does not require experienced users.

In other embodiments, ultrasound imaging probe 6 may include a 2D array of transducers to take 3D images of isolated organ 2. Even in that case, it may be useful to have ultrasound imaging system 8 control holder device 7 and ultrasound imaging probe 6 so as to take several 3D images, corresponding to different points of view or to different areas of isolated organ 3.

Ultrasound imaging system 8 is adapted to determine at least one quantitative index representing viability of isolated organ 2, said quantitative index being calculated from anatomical, tissue or vascular parameters obtained from ultrasound image(s) taken by ultrasound imaging probe 6.

For instance, once predetermined anatomic areas of isolated organ 2 have been automatically identified from said different images of the isolated organ 2 as explained above, said at least one quantitative index may be automatically computed in at least one area of interest among said predetermined anatomic areas. Said at least one area of interest may be manually chosen by an operator or may be automatically chosen or may be predetermined.

Said at least one quantitative index may be determined for several predetermined areas of isolated organ 2 and shown on a parametric map of said isolated organ. When isolated organ 2 is heart, said parametric map may be for instance a bullseye plot (polar plot) as defined by the American Heart Association (AHA), e.g. for showing the distribution of said at least one quantitative index across the left ventricle (for instance segmented according to the 17-Segment Model of the AHA).

### a) Quantitative Index Determined by Quantitative Shear Waves Elastography Imaging:

Said at least one quantitative index may be a rheological index of the tissues, determined by quantitative shear waves elastography imaging through ultrasound imaging probe 6 and ultrasound imaging system 8 as known in the art.

Said rheological index may be, for instance, stiffness, propagation speed of shear waves, fractional anisotropy, shear modulus or Young's modulus.

Said quantitative shear wave elastography imaging may be performed on the whole isolated organ or on selected structures.

Two examples will illustrate the relevance of quantitative shear wave elastography imaging to characterize the isolated organs, in the particular case where the isolated organ is the heart.

### Example 1 (FIG. 4)

In example 1, quantitative shear wave elastography imaging was performed on 10 anatomic sites of several porcine hearts, using the apparatus of FIGS. 1 and 2:
short-axis views of the left ventricular (LV) basal/mid/apex free walls,
right ventricular (RV) basal/mid/apex and mid septum,
and LV, RV, septum long-axis views.

The stiffness of these structures was monitored during 20 hours of storage at 4° C. on several hearts and an overall mean stiffness score is calculated (FIG. 4).

This example shows that the heart remains flexible during the first 4 hours of storage and then becomes much stiffer. This result is consistent with the maximum storage time of 4 hours used in clinical routine.

### Example 2 (FIG. 5)

Stiffness was also assessed for hearts that suffered before harvesting by warm ischemia, using the apparatus of FIGS. 1 and 2. The surgical model which was used here mimics the harvesting of so-called Maastricht-III hearts in humans. In the clinical setting, these are patients who have died after cardiac arrest but are potential organ donors. The major limitation preventing the use of organs from these patients is the initial uncontrolled suffering/alteration due to ischemia that may disqualify the graft. No means currently exist to characterize these grafts in the hypothermic situation.

FIG. 5 shows that these hearts are globally harder as soon as they are harvested and become even stiffer very quickly after 4 hours of conservation.

| | | | | Contractility | Relaxation | | SWV |
|---|---|---|---|---|---|---|---|
| | Preservation | RPP (1) | Myocardial | Rate | Rate | EDP (2) | (3) |
| Model | Time [h] | [mmHg/min] | Hardening | [mmHg/s] | [mmHg/s] | [mmHg] | [m/s] |
| SHAM | 4 | 14445.86 | 0 | 1908.29 | 1281.29 | 16.29 | 1.72 |
| SHAM | 20 | 10584.33 | 0.63 | 112.00 | 963.00 | 17.40 | 3.32 |
| ISCHEMIC | 4 | 4866.00 | 1.56 | 1259.00 | 945.00 | 15.50 | 3.31 |
| ISCHEMIC | 20 | 0.00 | 3.55 | 0.00 | 0.00 | 60.00 | 4.77 |

(1) Rate Pressure Product (RPP) = Heart Rate (HR) * Systolic Blood Pressure (SBP)
(2) End-Diastolic Pressure
(3) Shear Wave Velocity

Several hearts from both groups were resuscitated at 4 and 20 hours of preservation, and cardiac function was assessed with multifactorial scores.

SWV values correlated strongly with most measured function parameters. The highest correlation cardiac coefficient is found for Rate Pressure Product (RPP), the product between heart rate and systolic blood pressure used to determine the functional index (r\hat{ }2=0.86), myocardial hardening from palpation performed by two operators (r\hat{ }2=0.86) and relaxation rate (0.86). The other parameters also correlate well with SWV values, contractility rate which estimates contraction efficiency and end diastolic pressure (EDP) have an R-squared of 0.72 and 0.64.

| | | | Myocardial | Contractility | Relaxation | |
|---|---|---|---|---|---|---|
| | | RPP | Hardening | Rate | Rate | EDP |
| Pearson r | SWV | −0.93 | 0.93 | −0.85 | −0.93 | 0.80 |
| R squared | SWV | 0.86 | 0.86 | 0.72 | 0.86 | 0.64 |

### b) Other Tissue Properties:

Other tissue properties can used to characterize the heart:
elastic properties such as viscosity and elastic anisotropy, measured in particular as described for instance in WO2015/114232A1, or otherwise;
properties related to ultrasound backscatter: backscattered tensor imaging, backscattered energy etc.

Regarding the particular case of elastic anisotropy, when isolated organ 2 is fibrous (e.g. a heart), said at least one quantitative index may include a rheological elasticity parameter measured along fibers of isolated organ and a rheological elasticity parameter measured 2 perpendicular to said fibers. The rheological elasticity parameter in that case may be, for instance, the propagation speed of shear waves or any other rheological index mentioned above.

More precisely, such index may in that case be determined using a method for characterizing an anisotropic soft medium comprising at least one portion including fibers and having an outer surface, this method comprising the following steps:
(a) a measurement step during which at least one shear wave is generated which propagates by diverging from a central area in the anisotropic soft medium and, a propagation of said at least one shear wave is observed with ultrasonic observation transducers, from the surface of the anisotropic soft medium, in several predetermined propagation directions from said central area by maintaining fixed the ultrasonic observation transducers, said predetermined propagation directions comprising at least two directions forming between them an angle different from 0 degrees and different from 180 degrees, said ultrasonic observation transducers being positioned at least along said predetermined propagation directions and said measurement step being carried out within a period of less than 50 ms;
(b) at least one computing step during which at least one propagation parameter of the shear wave is determined, from data collected during the measurement step (a) in each of said predetermined propagation directions;
(c) a characterization step during which, from said at least one propagation parameter of the shear wave, determined in each of the propagation directions in the computing step (b), at least one rheological characteristic of the anisotropic soft medium is determined, selected from among a direction of the fibers of the anisotropic soft medium, a rheological elasticity parameter in a direction perpendicular to the fibers and a rheological elasticity parameter in the direction of the fibers. The rheological elasticity parameters determined during the characterization step (c) may be elasticity moduli.

### c) Vascular Properties:

Vascular properties of isolated organ 2 can also be quantified by ultrasound imaging through ultrasound imaging probe 6 and ultrasound imaging system 8. In that case, said isolated organ 2 is perfused in said organ preservation container 3, as known in the art. For this, it is useful to have ultrasound scatterers in the perfusion fluid (for instance red blood cells, ultrasound contrast agents such as microbubbles, nanobubbles, microdroplets or other molecular structures). Vascular flows can be imaged by Doppler imaging (using e.g. Power Doppler or pulsed Doppler) or by ultrasound localization microscopy (ULM) with microbubbles.

Quantitative parameters can thus be determined, such as flow, velocity, flow rate, blood volume, geometrical parameters of the vascular network such as vessel diameter (in particular micro-vessels), density and tortuosity of the vascular network.

### Example 3

Using the apparatus of FIGS. 1 and 2, ULM images of perfused porcine heart were made. FIG. 6 shows a mapping of the coronary arteries with a resolution of approximately 10 μm.

1. Method for ultrasonic evaluation of an isolated organ from a human or animal received in an organ preservation container made of an ultrasound transparent material, said method comprising:
holding an ultrasound imaging probe against said organ preservation container to perform ultrasound imaging of said isolated organ through said organ preservation container;
imaging said isolated organ by an ultrasound imaging system communicating with said ultrasound imaging probe, to obtain at least one ultrasound image of said isolated organ;
automatically determining at least one quantitative index representing viability of said isolated organ, said at least one quantitative index being calculated from anatomical, tissue and/or vascular parameters obtained from said at least one ultrasound image.

2. Method according to claim 1, wherein said ultrasound imaging probe is automatically moved by a holder device during said imaging, to obtain different images of said isolated organ as said ultrasound imaging probe is moved.

3. Method according to claim 2, wherein predetermined anatomic areas of said isolated organ are automatically identified from said different images and said at least one quantitative index is automatically computed in at least one area of interest among said predetermined anatomic areas.

4. Method according to claim 1, wherein said at least one quantitative index is determined for several predetermined areas of said isolated organ and shown on a parametric map of said isolated organ.

5. Method according to claim 1, wherein said isolated organ is chosen among a heart, a kidney and a liver.

6. Method according to claim 1, wherein said at least one quantitative index is a rheological index determined by elastography through said ultrasound imaging probe and ultrasound imaging system.

7. Method according claim 6, wherein said isolated organ includes fibers and said at least one quantitative index includes a rheological elasticity parameter measured along said fibers and a rheological elasticity parameter measured perpendicular to said fibers.

8. Method according to claim 1, wherein said at least one quantitative index is representative of vascular flows in the isolated organ under perfusion.

9. Method according to claim 1, wherein said at least one quantitative index is representative of vascular network geometry in said isolated organ under perfusion.

10. Method according to claim 8, wherein said isolated organ is perfused with a solution including a contrast agent adapted to enhance contrast for ultrasound imaging.

11. Method according to claim 1, wherein said at least one quantitative index is a property related to ultrasound backscatter.

12. Method according to claim 1, wherein said organ preservation container is made of an ultrasound transparent and rigid material.

13. Method according to claim 1, wherein said organ preservation container has a cylindrical shape adapted to perform multidirectional imaging of the isolated organ.

14. Apparatus for ultrasonic evaluation of an isolated organ from a human or animal, comprising:
an organ preservation container made of an ultrasound transparent material and adapted to contain said isolated organ;
an ultrasound imaging probe;
a holder device for holding the ultrasound imaging probe against said organ preservation container to perform ultrasound imaging of said isolated organ through said organ preservation container;
an ultrasound imaging system communicating with said ultrasound imaging probe to obtain at least one ultrasound image of said isolated organ, said ultrasound imaging system being adapted to automatically determine at least one quantitative index representing viability of said isolated organ, said at least one quantitative index being calculated from anatomical, tissue or vascular parameters obtained from said at least one ultrasound image.

15. Apparatus according to claim 14, wherein said holder device is adapted to automatically move said ultrasound imaging probe,
and wherein said ultrasound imaging system communicates with said holder device and is adapted to control said ultrasound imaging probe to obtain different images of said isolated organ as said ultrasound imaging probe is moved.

16. Apparatus according to claim 14, wherein said at least one quantitative index is a rheological index and said ultrasound imaging system is adapted to determine said rheological index by elastography.

17. Apparatus according to claim 14, wherein said at least one quantitative index is chosen among:
a quantitative index representative of vascular flows in the isolated organ;
a quantitative index representative of vascular network geometry in said isolated organ;
a property related to ultrasound backscatter.

18. Apparatus according to claim 14, wherein said organ preservation container is made of an ultrasound transparent and rigid material.

19. Apparatus according to claim 18, wherein said organ preservation container has a geometric shape adapted to perform multidirectional imaging of the isolated organ.
